(19) United States
(12) Patent Application Publication
(10) Pub. No.: US 2022/0354511 A1
(43) Pub. Date: Nov. 10, 2022

(54) THREE-DIMENSIONAL (3D) BONE-PROTECTING DRILL GUIDE DEVICE AND SYSTEMS AND METHODS OF MANUFACTURING AND USING DEVICE

(71) Applicant: Mazor Robotics Ltd., Caesarea (IL)

(72) Inventors: GAL BARAZANI, Haifa (IL); Ori Ben Zeev, Tel Aviv (IL); Ofir Dahan, Haifa (IL); Ziv Seemann, Beit Ytzhack (IL)

(73) Assignee: Mazor Robotics Ltd., Caesarea (IL)

(21) Appl. No.: 17/323,408

(22) Filed: May 18, 2021

Related U.S. Application Data
(63) Continuation-in-part of application No. 17/314,720, filed on May 7, 2021, now abandoned.

(51) Int. Cl. A61B 17/17; A61B 34/10; A61B 34/30; G16H 20/40; G16H 30/40; G16H 40/40; B33Y 80/00; B33Y 50/00

(52) U.S. Cl. CPC A61B 17/1757; A61B 34/10; A61B 34/30; G16H 20/40; G16H 30/40; G16H 40/40; B33Y 80/00; B33Y 50/00; A61B 2017/00526

(57) ABSTRACT

A surgical bone-protecting drill guide device includes a body formed of biocompatible material forming a shell. The body includes an outer surface, an interior surface being a reverse-engineering surface approximation of a protruding boney structure of one or more bones in an image of a patient and body material between the outer surface and the interior surface. The device includes implant guides. Each implant guide is configured to extend from the outer surface and through the body material and the interior surface and provide a window to a pre-planned implant location for implanting a respective one implant relative to the protruding boney structure of the patient. The window has a size and shape that is pre-calculated as a function of a size of a pre-determined tool to be inserted through the window.

### CROSS-REFERENCE TO RELATED APPLICATIONS

This application is a continuation-in-part of U.S. Ser. No. 17/314,720, entitled “THREE-DIMENSIONAL (3D) BONE-PROTECTING DRILL GUIDE DEVICE AND SYSTEMS AND METHODS OF MANUFACTURING AND USING DEVICE,” filed May 7, 2021, which is incorporated herein in its entirety.

### FIELD

The present technology is generally related to a three-dimensional (3D) bone-protecting drill guide device and systems and methods of manufacturing and using the device.

### BACKGROUND

Spinal disorders of the spine may result in symptoms, such as without limitation, nerve damage, and partial or complete loss of mobility and chronic pain. Surgical treatment of these spinal disorders includes correction, fusion, fixation, discectomy, laminectomy and implantable prosthetics, for example. As part of these surgical treatments, vertebral rods and bone fasteners are often used to provide stability to a treated region. During surgical treatment, a surgeon uses various surgical instruments to implant one or more rods and bone fasteners to a surgical site.

During surgery, in certain situations, an instrument is mounted or clamped to a boney structure. The instrument may have a rigidity that allows it to deflect more under a load as compared to a stiffer construction. However, mounting instruments to boney structures can cause surface damage to the boney structure, which can affect recovery. In other instances, the boney structure in certain patients can be soft or brittle, making the boney structure a less than optimum support structure for the mounting or clamping of a surgical instrument. Still further, determining a support structure can be a challenge for revision surgery due to missing boney structures.

This disclosure describes an improvement over these prior art technologies.

### SUMMARY

The techniques of this disclosure generally relate to a three-dimensional (3D) bone-protecting drill guide device and systems and methods to manufacture and use a 3D bone-protecting drill guide device to, for example, provide bone protection on a portion of a boney structure of at least one bone and overlay at least one pre-planned implant guide.

In one aspect, the present disclosure provides a surgical bone-protecting drill guide device having a body formed of biocompatible material forming a shell. The body may include an outer surface, an interior surface being a reverse-engineering surface approximation of a protruding boney structure of one or more bones in an image of a patient and body material between the outer surface and the interior surface. The device may include implant guides. Each implant guide is configured to extend from the outer surface and through the body material and the interior surface and provide a window to a pre-planned implant location for implanting a respective one implant relative to the protruding boney structure of the patient. The window has a size and shape that is pre-calculated as a function of a size of a pre-determined tool to be inserted through the window.

In another aspect, the disclosure provides method that includes receiving, by a computing system, pre-operative image data of at least one bone with a protruding boney structure of a patient; receiving, by the computing system, pre-planned implant location data of pre-planned implant locations at which implants are to be implanted relative to the protruding boney structure of the at least one bone; and modeling, by the computer system, a body of a three-dimensional bone-protecting drill guide device. The modeling, by the computer system, may include forming an interior surface as a reverse-engineering surface approximation of the protruding boney structure of the patient; forming an outer surface having a predetermined thickness from the interior surface, and forming implant guides. Each implant guide may be configured to extend from the outer surface and through the body and the interior surface. The implant guides provide a window to the pre-planned implant location for implanting a respective one implant relative to the protruding boney structure of the patient. The window has a size and shape pre-calculated a function of a size of a pre-determined tool to be inserted through the window.

In another aspect, the disclosure provides a method that includes providing a bone-protecting drill guide device for a protruding boney structure of a patient; installing the bone-protecting drill guide device on the protruding boney structure of the patient; registering a location of an implant guide; mounting a surgical instrument to the installed bone-protecting drill guide device; and drilling a hole for a bone construct using the implant guides using the mounted surgical instrument.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a block diagram that illustrates an example computing system for generating a model of a 3D bone-protecting drill guide device.

FIG. 2 is a flow chart that illustrates an example method to create a 3D bone-protecting drill guide device.

FIG. 3 is a prior art diagram that illustrates an example graphical user interface to display a medical images of a patient bone and select a treatment area for planning a surgery.

FIG. 4 is a prior art diagram that illustrates an example graphical user interface to enlarge and display vertebrae of the treatment area for planning the surgery.

FIG. 5 is a diagram that illustrates an example graphical user interface to select vertebrae in the treatment area to generate a model of a 3D bone-protecting drill guide device during planning the surgery.

FIG. 6A is a prior art diagram that illustrates an example graphical user interface to select a position areas for implanting a bone construct during planning a surgery.

FIG. 6B is a prior art diagram that illustrates an example graphical user interface to adjust a position to implant a bone construct during planning a surgery.

FIG. 7 is a diagram that illustrates an example graphical user interface representative of a planned layout to graphical define location and size of the 3D bone-protecting drill guide device over the boney structure of selected vertebrae of the treatment area and the implant guide.

FIG. 8 is an exploded end view that illustrates an example 3D bone-protecting drill guide device having at least one closed end and mounted on the boney structure of a cervical vertebra of a cervical treatment area.

FIG. 9A is a perspective view that illustrates an example 3D bone-protecting drill guide device having at least one open end.

FIG. 9B is a perspective view that illustrates an example 3D bone-protecting drill guide device for protecting two adjacent vertebrae and having at least one open end.

FIG. 10 is a perspective end view that illustrates an example 3D bone-protecting drill guide device having a two-piece construction for a boney structure of a thoracic vertebra of a thoracic treatment area.

FIG. 11A is a perspective view that illustrates an example 3D bone-protecting drill guide device having at least one open end for a boney structure of a thoracic vertebra of a thoracic treatment area.

FIG. 11B is a perspective view that illustrates an example 3D bone-protecting drill guide device of FIG. 11A with a robotic effector interface.

FIG. 12A is a perspective view that illustrates an example 3D bone-protecting drill guide device having a two-piece construction for a boney structure of a lumbar vertebra of a lumbar treatment area.

FIG. 12B is a perspective view that illustrates an example 3D bone-protecting drill guide device having a one-piece construction installed on a boney structure of a lumbar vertebra.

FIG. 13 is a flowchart that illustrates a method of performing surgery using the 3D bone-protecting drill guide device.

FIG. 14A is a diagram that illustrates a surgical implant system employing the computing system of FIG. 1.

FIG. 14B is a diagram that illustrates a robotic arm and end effector of the robotic guidance system for connection to the 3D bone-protecting drill guide device of FIG. 11B.

FIG. 15 is a flow diagram that illustrates a method for the production of a 3D bone-protecting drill guide device.

FIG. 16 depicts an example of internal hardware that may be included in any of the electronic components of an electronic device.

### DETAILED DESCRIPTION

The embodiments of the 3D bone-protecting drill guide devices may be used to protect boney structures of a treatment area from damage due to mounting or clamping an instrument to a boney structure and/or impact forces during certain phases of a surgery to treat a bone or joint. In some embodiments, the 3D bone-protecting drill guide device may be used in a surgery with the purpose of implantation of bone constructs for the treatment of musculoskeletal disorders and more particularly, in terms of a surgical system and a method for treating a spine.

In various embodiments, a surgical implant system may include a 3D bone-protecting drill guide device to, for example, provide bone protection on a portion of the boney structure of at least one vertebra and overlay pre-planned implant guides on at least one vertebra to locate registered locations for drilling into the at least one vertebra, and the related methods of use that can be employed with drills or other instruments for implanting spinal constructs including bone fasteners and connectors of a surgical implant system for spine surgeons.

In various embodiments, a surgical system may include a 3D bone-protecting drill guide device to, for example, provide bone protection on a portion of a boney structure of at least one bone or joint and overlay pre-planned implant guides on at least one bone or joint to locate registered locations for drilling into the bone or joint, and the related methods of use that can be employed with drills for drilling holes for implanting bone constructs including bone fasteners and connectors that provide a surgical system for surgeons. The bone or joint, may include, for example, a knee, hip, shoulder, elbow, and ankles.

The embodiments of the surgical implant system may be used for various approaches to fixation as an adjunct to fusion for the following indications: degenerative disc disease (defined as back pain of discogenic origin with degeneration of the disc confirmed by history and radiographic studies), spondylolisthesis, trauma (i.e., fracture or dislocation), spinal stenosis, curvatures (i.e., scoliosis, kyphosis, or lordosis), tumor, pseudarthrosis, knee fusion, and/or failed previous fusion. The surgical implant system may be used for cervical segment surgery, thoracic segment surgery, and lumbar segment surgery. The surgical implant system may be used in pediatric spine surgery.

The surgical implant system of the present disclosure may be understood more readily by reference to the following detailed description of the embodiments taken in connection with the accompanying drawing figures that form a part of this disclosure. It is to be understood that this application is not limited to the specific devices, methods, conditions or parameters described and/or shown herein, and that the terminology used herein is for the purpose of describing particular embodiments by way of example only and is not intended to be limiting. Also, in some embodiments, as used in the specification and including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment. It is also understood that all spatial references, such as, for example, horizontal, vertical, top, upper, lower, bottom, front, back, left and right, are for illustrative purposes only and can be varied within the scope of the disclosure. For example, the references “upper” and “lower” are relative and used only in the context to the other, and are not necessarily “superior” and “inferior”.

Further, as used in the specification and including the appended claims, “treating” or “treatment” of a disease or condition refers to performing a procedure that may include administering one or more drugs to a patient (human, normal or otherwise or other mammal), employing implantable devices, and/or employing instruments that treat the disease, such as, for example, microdiscectomy instruments used to remove portions bulging or herniated discs and/or bone spurs, in an effort to alleviate signs or symptoms of the disease or condition.

Alleviation can occur prior to signs or symptoms of the disease or condition appearing, as well as after their appearance. Thus, treating or treatment includes preventing or prevention of and/or reducing the likelihood of a certain disease or undesirable condition (e.g., preventing or reducing the likelihood of the disease from occurring in a patient, who may be predisposed to the disease but has not yet been diagnosed as having it). In addition, treating or treatment does not require complete alleviation of signs or symptoms, does not require a cure, and specifically includes procedures that have only a marginal effect on the patient. Treatment can include inhibiting the disease, e.g., arresting its development, or relieving the disease, e.g., causing regression of the disease. For example, treatment can include reducing acute or chronic inflammation; alleviating pain and mitigating and inducing re-growth of new ligament, bone and other tissues; as an adjunct in surgery; and/or any repair procedure. Also, as used in the specification and including the appended claims, the term “tissue” includes soft tissue, ligaments, tendons, cartilage and/or bone unless specifically referred to otherwise.

The following discussion includes a description of a computing system for generating a model of a 3D bone-protecting drill guide device, a system for 3D printing or manufacturing a 3D bone-protecting drill guide device, a surgical implant system including 3D bone-protecting drill guide device, and methods of employing the surgical system in accordance with the principles of the present disclosure. Alternate embodiments are also disclosed. Reference is made in detail to the exemplary embodiments of the present disclosure, which are illustrated in the accompanying figures.

The 3D bone-protecting drill guide device can be fabricated from biologically acceptable materials suitable for medical applications, including computer aided metals, computer aided plastics, metals, synthetic polymers, ceramics and bone material and/or their composites. For example, the 3D bone-protecting drill guide device can be fabricated from materials such as stainless steel alloys, aluminum, commercially pure titanium, titanium alloys, Grade 5 titanium, super-elastic titanium alloys, cobalt-chrome alloys, stainless steel alloys, superelastic metallic alloys (e.g., Nitinol, super elasto-plastic metals, such as GUM METAL® manufactured by Toyota Material Incorporated of Japan), ceramics and composites thereof such as calcium phosphate (e.g., SKELITE™ manufactured by Biologic, Inc.), thermoplastics such as polyaryletherketone (PAEK) including polyetheretherketone (PEEK), polyetherketoneketone (PEKK) and polyetherketone (PEK), carbon-PEEK composites, PEEK-BaSO4 polymeric rubbers, polyethylene terephthalate (PET), fabric, silicone, polyurethane, silicone-polyurethane copolymers, polymeric rubbers, polyolefin rubbers, hydrogels, semi-rigid and rigid materials, elastomers, rubbers, thermoplastic elastomers, thermoset elastomers, elastomeric composites, rigid polymers including polyphenylene, polyamide, polyimide, polyetherimide, polyethylene, epoxy, bone material including autograft, allograft, xenograft or transgenic cortical and/or corticocancellous bone, and tissue growth or differentiation factors, partially resorbable materials, such as, for example, composites of metals and calcium-based ceramics, composites of PEEK and calcium based ceramics, composites of PEEK with resorbable polymers, totally resorbable materials, such as, for example, calcium based ceramics such as calcium phosphate, tri-calcium phosphate (TCP), hydroxyapatite (HA)-TCP, calcium sulfate, or other resorbable polymers such as polyaetide, polyglycolide, polytyrosine carbonate, polycaroplaetohe and their combinations.

While certain embodiments described herein are directed to spines and the bones of a spine, the 3D bone-protecting drill guide device has application for other bones with boney structures, which are subject to damage due to clamping or mounting surgical instruments or impact forces during surgery by surgical instruments. The 3D bone-protecting drill guide device can be used to reduce the registration time during surgery for forming a drill guide to indicate an entry point to drill holes in which to implant one or more bone constructs into a bone or joint. The 3D bone-protecting drill guide device has application during surgery to protect soft and/or brittle bones. The 3D bone-protecting drill guide device has application to create a temporary prosthetic of any missing boney structures that would be needed to conduct a surgery.

The 3D bone-protecting drill guide device may have material composites, including the above materials, to achieve various desired characteristics such as strength, rigidity, elasticity, compliance, biomechanical performance, durability and radiolucency or imaging preference. The 3D bone-protecting drill guide device may also be fabricated from a heterogeneous material such as a combination of two or more of the above-described materials. The 3D bone-protecting drill guide device may be monolithically formed, as described herein.

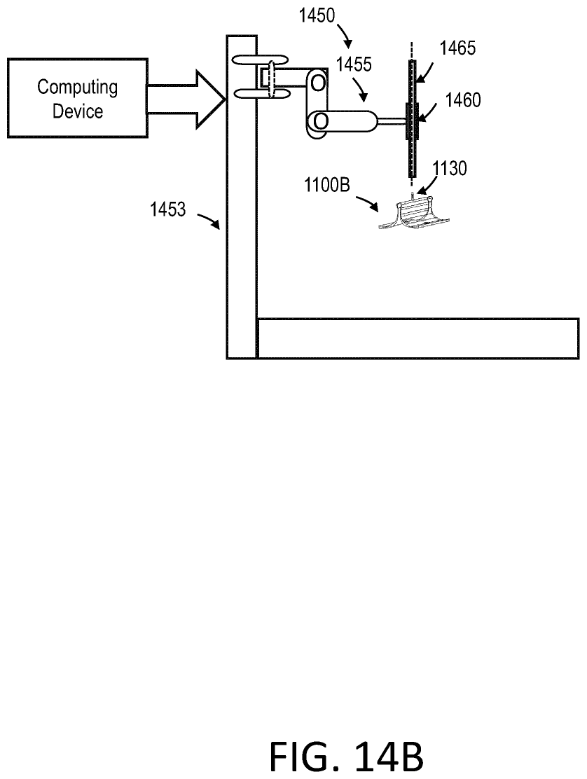

The surgical implant system 1400 (FIG. 14A) may include, for example, the Mazor X Stealth™ Edition robotic guidance system, by Medtronic Sofamor Danek USA, Inc. The surgical implant system 1400 may be employed, for example, with an open, minimal access and/or minimally invasive including percutaneous surgical technique to deliver and fasten an implant at a surgical site within a body of a patient, for example, a section of a spine. In one embodiment, the 3D bone-protecting drill guide device may be configured to provide bone protection and a pre-planned implant guide to assist in registering locations for drilling holes to implant and/or fix a bone fastener, such as a pedicle screw, or other implants within tissue for a surgical treatment to treat various spine pathologies, such as those described herein.

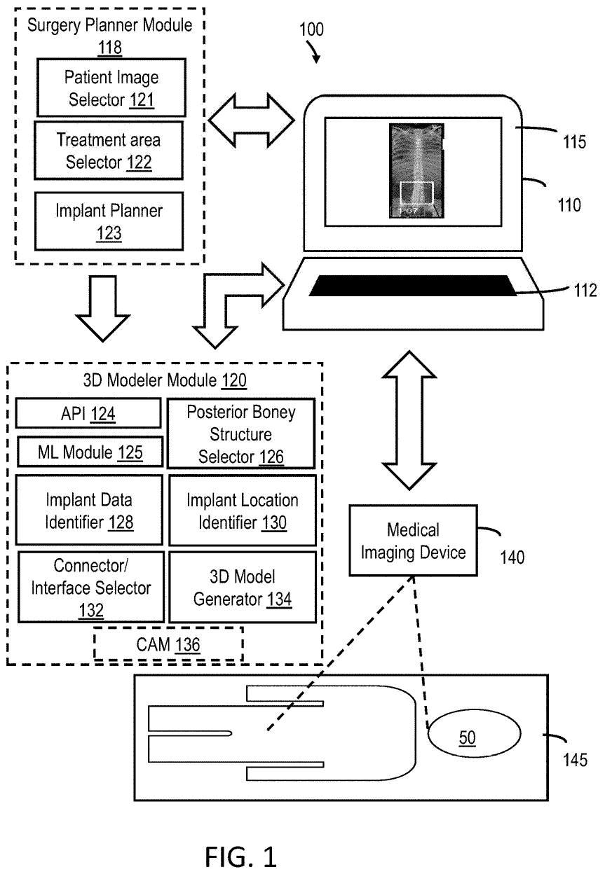

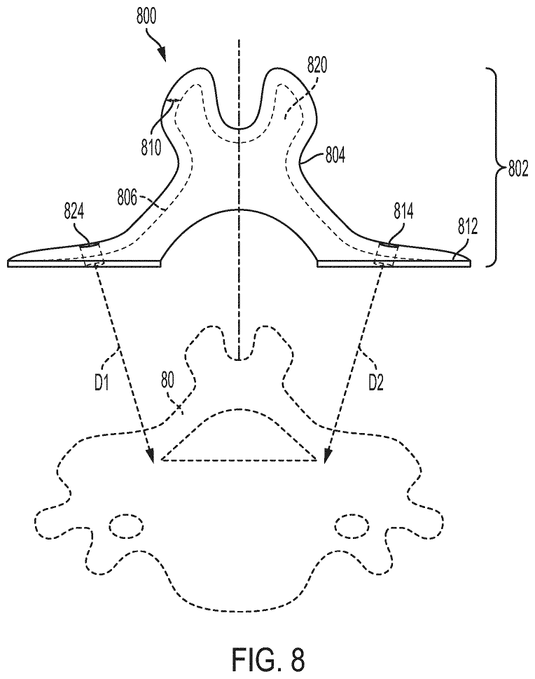

Embodiments directed to the generation of the 3D bone-protecting drill guide device will be described in relation to FIGS. 1-7. Example, 3D bone-protecting drill guide devices will be described in relation to FIGS. 8-12A-B. The 3D bone-protecting drill guide device has application to apply minimal stress to the patient's boney structures due to multiply contact surfaces. This may have benefits to during healing time but also reduce the risk of damage (spinous process brake) and improve general rigidity (lower deflection under load). In some scenarios, the 3D bone-protecting drill guide devices may eliminate the need to connect to the Sacrum during certain spine surgeries.

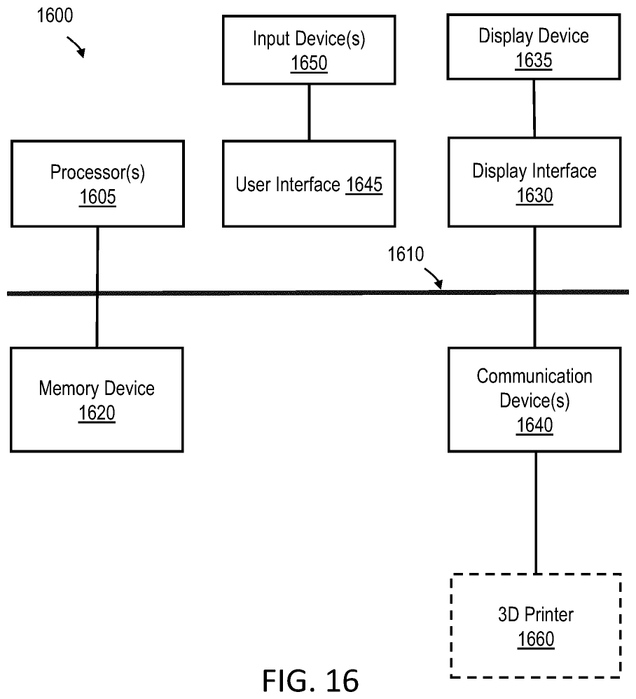

FIG. 1 is a block diagram that illustrates an example computing system 100 for creating a 3D bone-protecting drill guide device. The computing system 100 may interface with the surgical implant system 1400 (FIG. 14A) or be part of the surgical implant system 1400. The computing system 100 may include a computing device 110 having a display device 115 configured to display medical images from a medical imaging device 140. The computing system 100 may include at least one user input device 112. The computing device 110 will be described in more details in relation to FIG. 16. The computing device 110 may interface with a medical imaging device 140. The medical imaging device 140 may include a computed tomography (CAT) scanner, a magnetic resonance imaging (MRI) device and a high-energy electromagnetic radiation (X-ray) device. The medical imaging device 140 may be part of the computing system 100 or its own standalone device, where the computing system 100 may receive the imaging data directly or indirectly from the medical imaging device 140. For example, the imaging data may be stored on a server or in a cloud.

In some embodiments, the medical imaging device 140 and the computing device 110 may be part of the surgical implant system 1400 (FIG. 14A). The medical imaging device 140 may be configured to capture pre-operative image data of at least one bone of a patient 50 while lying on a table 145. In other embodiments, the medical imaging device 140 may be configured to capture images of at least one bone of a patient 50 while the patient is standing. The medical imaging device 140 may capture multiple view from different perspectives. For example, the medical imaging device 140 may capture posterior views and anterior views of the coronal planes, sagittal planes and transverse planes.

Each patient has a unique anatomical boney structure. The anatomical honey structure may be deformed such that it diverges from the normal. A patient may have experienced a previous surgery with certain anatomical boney structures removed or altered. Still further, a patient may have experienced an accident that caused trauma to the anatomical boney structure. The images may capture these deformities or defects for use by the surgeon when planning a surgery.

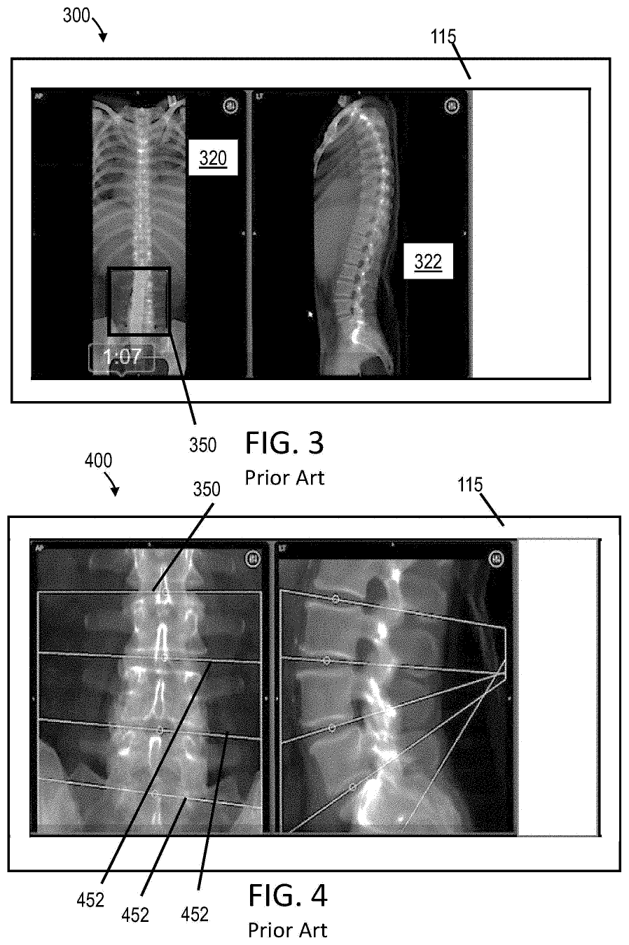

The computing system 100 may include applications including an operating system. The computing system 100 may include a surgery planner module 118. The surgery planner module 118 may be implemented using hardware, firmware, software or a combination of any of these. For instance, surgery planner module 118 may be implemented as part of a microcontroller, processor, and/or graphics processing units (GPUs) of computing device 110. The surgery planner module 118 may include or interface with a register, data store or memory device 1620 (FIG. 16) for storing data and programming instructions, which when executed, plans a surgery, such as for implantation of a bone construct, such as a bone fastener. The surgery planner module 118 may include programming instructions that include graphical user interfaces, as will be described in relation to FIGS. 3, 4 and 6A-6B. The surgery planner module 118 may be used for pre-planning surgical processes in advance of performing a surgery performed either by a robotic device of the surgical implant system 1400 (FIG. 14A) and/or the surgeon.

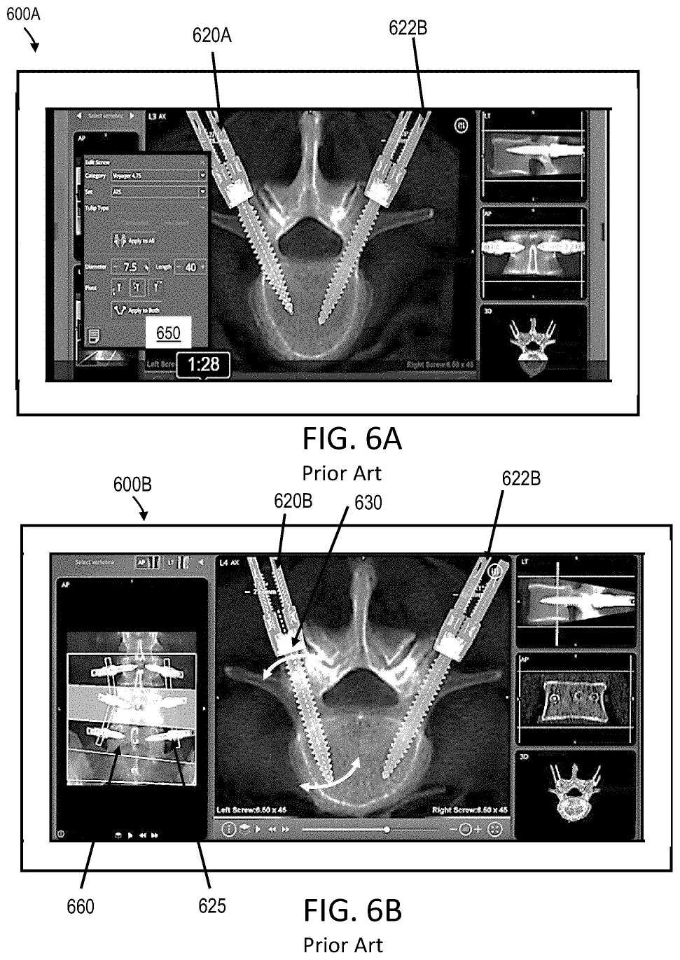

The computing system 100 may include a modeler module 120. The modeler module 120 may be implemented using hardware, firmware, software or a combination of any of these. For instance, the modeler module 120 may be implemented as part of a microcontroller, processor, and/or graphics processing units (GPUs) of computing device 110. The modeler module 120 may include or interface with a register, data store or memory device 1620 (FIG. 16) for storing data and programming instructions, which when executed, generates a computer-generated model of a 3D bone-protecting drill guide device based on the surgery pre-plans for implantation of a bone construct, and an implant pre-plan guide of registered implant positions in the image of the patient spine, as will be discussed in relation to FIGS. 6A and 6B. The computer-generated model of a 3D bone-protecting drill guide device may be generated in computer-aided design (CAD) software, for example, via 3D model generator 134. Other examples of computer modeling software may include AutoCAD, SolidWorks, CATIA, to name a few. The computer modeling software may interface with a computer-aided manufacturing (CAM) application 136 to manufacture the 3D bone-protecting drill guide device, as will be discussed later.

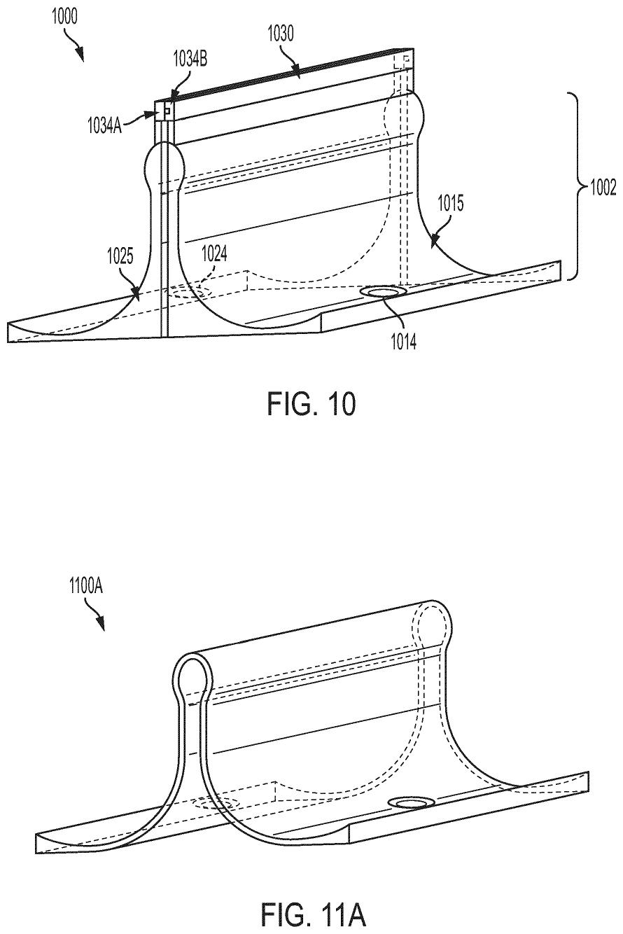

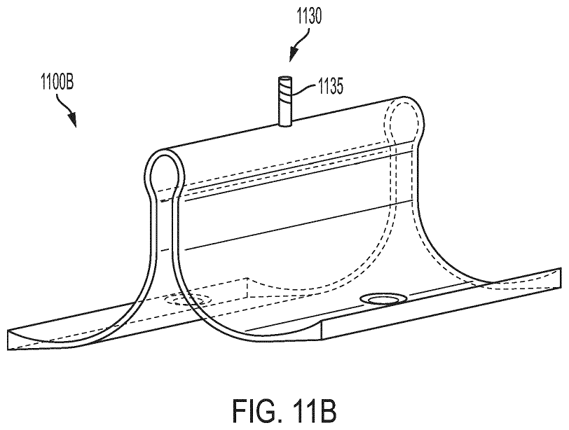

The modeler module 120 may include a connector/interface selector 132. The modeler module 120 may provide the user a graphical user interface to select a connector type, as shown in FIG. 10, for example. An example, robotic effector interface is shown in FIGS. 11B and 14B.

In some embodiments, the computing system 100 may interface with a 3D production unit directly or indirectly, as will be described in relation to FIG. 16.

The surgery planner module 118 may include a patient image selector 121 to obtain the at least one captured image of at least one bone of the patient 50. The patient image selector 121 may include programming instructions for displaying a graphical user interface 300 (FIG. 3). The surgery planner module 118 may include a treatment area selector 122 that includes an interface to receive input and selection from a user input device 112, such as, a mouse, a keyboard, a joystick, a touch screen, a remote control, or a pointing device. The surgery planner module 118 may include an implant planner 123 configured to allow a surgeon to plan the implantation of at least one bone construct, such as a pedicle fastener, to each selected vertebra in the treatment area 350, as will be described in relation to FIGS. 6A-6B.

FIG. 3 is a prior art diagram that illustrates an example graphical user interface 300 to display on display device 115 at least one captured medical image 320 of at least one bone of the patient, and to select a treatment area 350 for planning a surgery, such as available by the Mazor X Stealth™ Edition robotic guidance system, by Medtronic Sofamor Danek USA, Inc. The surgery planner module 118 retrieves imaging data or images 320 of at least one patient bone, such as the spine. Using a mouse or other user input device, the user may select a treatment area 350, denoted in a highlighted box, using the treatment area selector. In this example, a set of lumbar vertebrae are selected. Additionally, the example graphical user interface 300 may display two medical images 320 and 322 side-by-side of multiple views of the same anatomical area, bone(s) or boney structure(s).

FIG. 4 is a prior art diagram that illustrates an example graphical user interface 400 to enlarge and display on display device 115 the treatment area 350 and related vertebrae for planning the surgery, such as available by the Mazor X Stealth™ Edition robotic guidance system, by Medtronic Sofamor Danek USA, Inc. In FIG. 4, a posterior side of the vertebrae is shown in the graphical user interface 400. In the example, the lines 452 divide the vertebras in the treatment area 350.

FIG. 6A is a prior art diagram that illustrates an example graphical user interface 600A to select position areas for implanting bone constructs 620A and 622A of a single vertebra during planning a surgery, such as available by the Mazor X Stealth™ Edition robotic guidance system, by Medtronic Sofamor Danek USA, Inc. The graphical user interface 600A provides a position adjustor tool 650 to adjust the position of the bone construct.

FIG. 6B is a prior art diagram that illustrates an example graphical user interface 600B to adjust a position to implant the bone constructs 620B and 622B of a single vertebra during planning a surgery such as available by the Mazor X Stealth™ Edition robotic guidance system, by Medtronic Sofamor Danek USA, Inc. As can be seen from the graphical user interface 600B, the surgery planning is able to adjust each bone construct individually for the treatment of the spine. The arrow 630 represents an example range of movement adjustment of the bone constructs 620A through the pedicle of the selected one vertebra. The window 660 illustrates the pre-planned positioning of a plurality of bone constructs 625, such as bone constructs 620B and 622B, in three vertebra. A user may select one or more of these vertebrae to form the 3D bone-protecting drill guide device.

The 3D bone-protecting drill guide device may be used to prevent the formation of bone damage from impact forces generated by a surgical instrument during a surgery. The surgeon when planning the surgery can determine which boney structures in the treatment are may be needed for mounting or clamping to perform the surgery. By way of non-limiting example, mounting can be done by generating a robotic/navigation interface on the device for interconnection with the robotic/navigation device. The device may be used as a bone protecting over the boney structures before mounting or clamping a surgical instrument. Additionally, the surgeon may determine which bones or vertebrae will have a bone implant.

The 3D bone-protecting drill guide modeler module 120 may include an application programming interface (API) 124 to obtain information from the surgery planner module 118 to generate a model of a 3D bone-protecting drill guide device via 3D model generator 134 based on the entered and/or received data. The modeler module 120 may include boney structure selector 126 that allows a user using an input device 112 to select those vertebrae to be modeled for the modeler module 120.

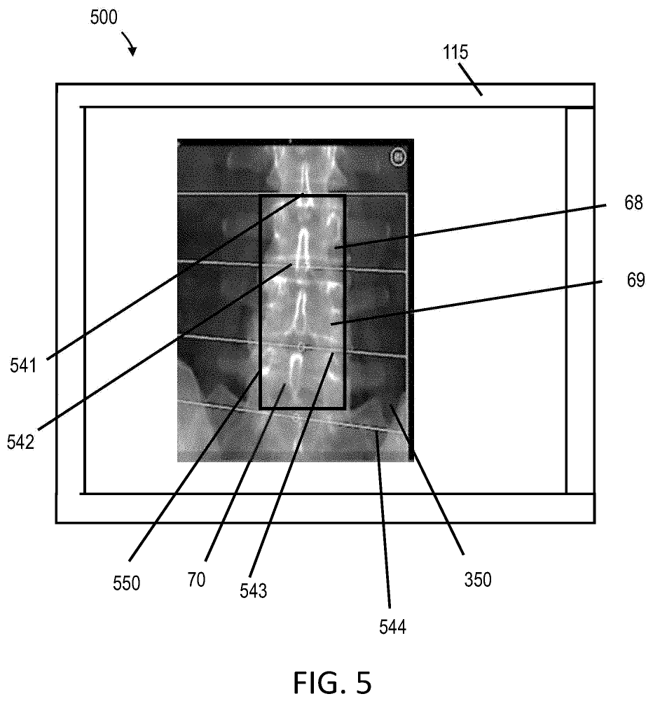

FIG. 5 is a diagram that illustrates an example graphical user interface 500 displayed on display 115 to select those vertebrae 68, 69, and 70 in the treatment area 350 to generate a model of a 3D bone-protecting drill guide device during planning the surgery. The API 124 may be configured to retrieve the treatment area 350 defined by the surgery planner module 118. In other embodiments, the graphical user interface 500 may generate its own treatment area.

The highlighted area 550 may include those selected vertebrae 68, 69, 70 on which the 3D bone-protecting drill guide device will be installed. In various embodiments, the highlighted area 550 may include those vertebrae that will have implanted bone constructs. The graphical user interface 500 may include lines 542, 543 and 544 to denote the demarcation between each vertebra 68, 69, and 70. In some embodiments, a monolithic 3D bone-protecting drill guide device may be installed on a plurality of vertebrae. In other embodiment, each vertebra 68, 69, and 70 may have its own 3D bone-protecting drill guide device, each demarcated to be within the lines 542, 543 and 544

Returning again to FIG. 1, the modeler module 120 may include an implant data identifier 128. The implant data identifier 128 may include the type and size of the implant and/or implant drill type. In some embodiments, the implant data identifier 128 may identifier the type of drill to be used so that the holes of the implant guide has an opening or diameter to receive a drill implement. For example, the implant guide may have guide indicators 2 or 4 times the diameter of the drill implement. The guide hole diameter may be in the range of 1.2-5 times the diameter of the drill implement.

The modeler module 120 may include an implant location identifier 130. By way of non-limiting example, the API 124 may access the information associated with the locations and angles of the plurality of bone constructs 625, planned by the surgery planner module 118, as shown in widow 660 of FIG. 6B, to form the implant guides.

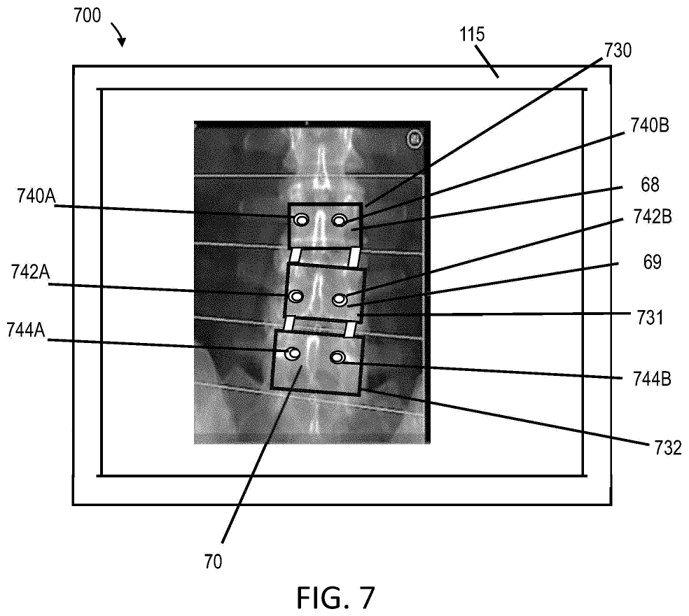

FIG. 7 is a diagram that illustrates an example graphical user interface 700A representative of a planned layout to graphical define a location and size of the 3D bone-protecting drill guide device over the boney structure of the selected vertebrae of the treatment area and the implant guide. The boney structure areas 730, 731 and 732 of vertebrae 68, 69 and 70, respectively, may be selected by a surgeon or by machine-learning for the creation of the 3D bone-protecting drill guide device using a reverse-engineering surface approximation. For example, a mesh may be generated on the surface to generate a 3D model of the surface in the image. For example, applications, such as Solidworks, FreeCAD, BlocksCAD, AutoCAD, OpenSCAD, Pro/E, TinkerCAD, Fusion360°, Rhinoimatron, Solid Edge, Unigrafics, Mesh2Surface, CYBORG3D MeshToCAD or other reverse-engineering surface software may be used. Although the example shown three areas 730, 731 and 732, the graphical user interface 700A may form a single area around the three selected vertebrae.

The computer system 100 may include machine-learning (ML) module 125 with ML models and algorithms. The ML models may be generated based on medical images for a plurality of individuals. Each ML model represents a possible structure of a body part (e.g., a spine). In this regard, each ML model may define relative locations of femoral heads to vertebrae, relative locations of vertebrae to each other, dimensions of vertebrae, relative locations of vertebrae edges, angles of vertebrae edges relative to a reference line, a centerline of the spine, a curvature of the centerline and boney structures of the vertebrae or other bone. The ML model may be of other boney structures of other bones and joints.

The computing system 100 may perform ML algorithms employing feature extraction algorithms for detecting an object, such as boney structures. The feature extraction algorithms may include, without limitation, edge detection, corner detection, template matching, dynamic texture processing, segmentation image processing, object recognition and classification, etc. For example, in a scenario for the treatment of the spine, a treatment area may include at least one of a cervical segment, a thoracic segment or a lumbar segment. The at least one protruding boney structure may include at least one of a spinous process, a transverse process, an articular process, an inferior articular process, and/or a superior articular process. The at least one adjacent boney structure may include a vertebra lamina between two adjacent protruding boney structures. The machine-learning algorithm may distinguish the boney structures of the vertebrae in the imaging data. For example, in the thoracic segment, the boney structures of the thoracic vertebrae may not have articular processes found in the boney structures of the lumbar vertebrae. The machine-learning algorithm may detect those vertebrae in the cervical segment because the boney structures of the cervical vertebrae may not have transverse processes. Additionally, the spinous process of the cervical segment have distinguishing features when compared to the spinous process of the thoracic vertebrae. Still further, the honey structures of the lumbar vertebrae also has unique features distinguishable from the cervical vertebrae and the thoracic vertebrae. The ML algorithm may distinguish each level of vertebrae in each segment of the spine.

The ML algorithms may employ supervised ML, semi-supervised ML, unsupervised ML, and/or reinforcement ML. Each of these listed types of machine-learning algorithms is well known in the art.

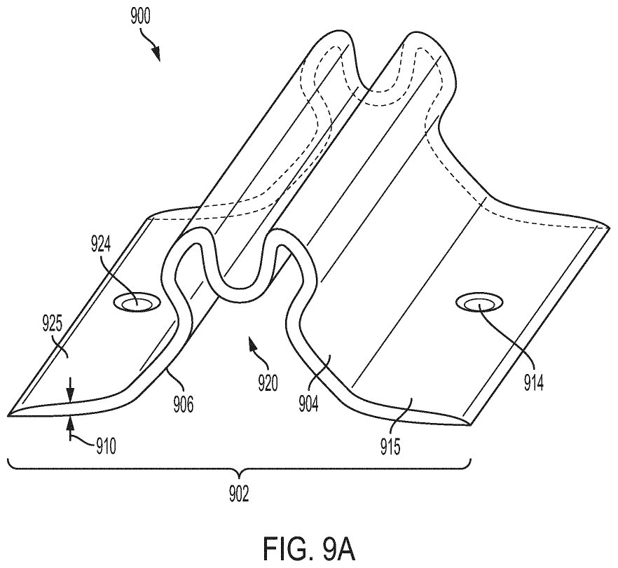

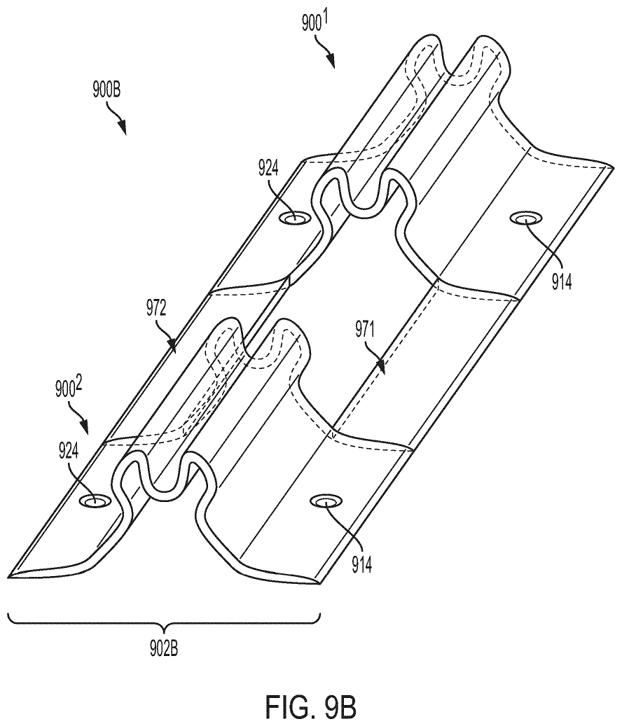

The dimensions of each boney structure areas 730, 731 and 732 may be defined to include an implant guide pad 760 to the lateral sides of the spinous process, which may overlap some or all of the lamina of a vertebra. The implant guide pad 760 provides an area to form the implant guide for each level of the spine for drilling into the vertebra or the pedicle during surgery. Forming a single 3D bone-protecting drill guide device for two or more vertebrae allows pre-registration of the guides 740A, 742A, 744A, 740B, 742B and 744B relative to the other guides. Hence, during surgery, once a single guide hole in the implant guide is registered, the other guide holes become registered. An example of a single 3D bone-protecting drill guide device with two vertebral levels is shown in FIG. 9B.

The implant guides may be placed to align with locations associated with the pedicle of the vertebra, such as at a location drilling for implantation of a bone construct represented in window 660. A single 3D bone-protecting drill guide device to be installed may vary in dimensions along each level of the vertebral column.

In FIG. 7, each implant guide (e.g., guides 740A, 742A, 744A, 740B, 742B and 744B) is configured to extend from the outer surface and through the body material and the interior surface, as will be discussed in FIG. 8. The implant guide may provide a window to a planned implant location relative to the at least one boney structures of the patient. The implant guide may also define an angle at which a drill implement should be inserted to create the angle at which the threads and point of a pedicle fastener will be implanted. In the illustrations, the guides 740A, 742A, 744A, 740B, 742B and 744B are represented for the purposes of discussion with a center axis being offset in the direction of drilling. Each guide hole offset may be different from any other guide hole.

The modeler module 120 may include a computer-aided manufacturing (CAM) application 136 to interface with a 3D printer device 1660 (FIG. 16) or other CAM device. The CAM application 136 is shown in a dash, dot box to denote that it is optional. The manufacturing of the 3D bone-protecting drill guide device may be performed remote from the computing system 100 or a vendor.

The 3D printer may print a reverse counter surface, such as without limitations, a mold of the selected boney structure with the implant pad with discrete guides that identify placement of the drill to pass through. In some applications, the 3D model generated by the 3D model generator 134 may be sent to a vendor that can print the 3D bone-protecting drill guide device based on the 3D model.

The graphical user interface 700A may receive user input representative of acceptance of the layout of the 3D bone-protecting drill guide device. The graphical user interface 700A may allow other features to be selected and pre-placed such as connectors and placement of connectors, as will be discussed in more detail in relation to FIG. 10.

In the scenario of FIG. 7, each vertebra is shown with a spinous process. However, in some instances, a vertebra may be deformed or damaged. For example, a spinous process may be damaged and not available to directly create a reverse-engineering surface of the spinous process of a vertebral column level.

The bone-protecting drill guide device has application for use in a revision surgery, such as a second spine surgery. For example, if a spinous process was previously removed, there may be no mounting area to revise the surgery. In this scenario, surgeon or other user may make a model of a bone-protecting drill guide device that may be used to extend bone protection from adjacent spinous processes or other boney structures of adjacent vertebral levels over the area of the missing spinous process. The 3D bone-protecting drill guide device may be configured to provide bone protection over the vertebrae without a spinous process or other boney structure to which a surgical instrument may be mounted or clamped.

In some embodiments, a 3D model of missing boney structures needed for the model of the 3D bone-protecting drill guide device may be generated using one or more of: 1) 3D models of a plurality of boney structures of the same class; or 2) imaging data of other boney structures of the same class of the patient. A class may be a bone or joint type.

In various embodiments, a 3D bone-protecting drill guide device for a level of the vertebral column with a missing spinous process, for example, may still be created for use in mounting or clamping an instrument, implant registration and bone protection.

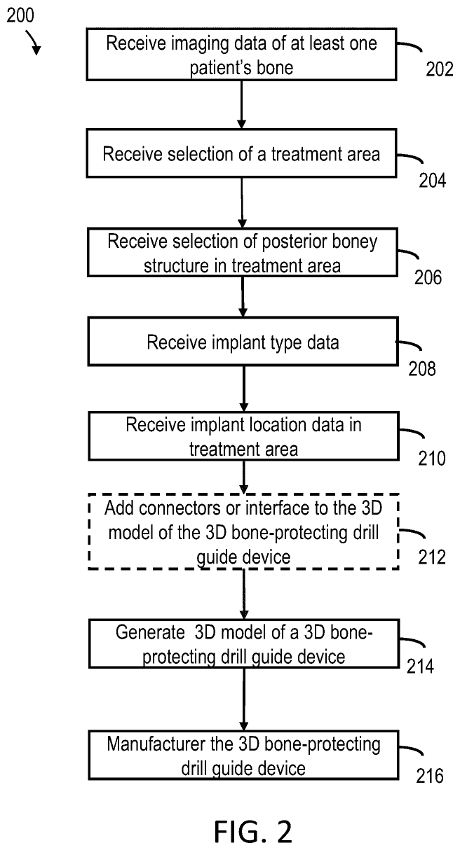

FIG. 2 is a flow chart that illustrates an example method 200 to create a 3D bone-protecting drill guide device. The steps of the method may be performed in the order shown or a different order. One or more steps may be performed contemporaneously. One or more steps may be deleted or other steps added.

The method 200 may include, by the computing system 100, receiving image data (at 202) of at least one patient's bone using a graphical user interface (e.g., graphical user interface 300 of FIG. 3); and receiving data representative of a selection in the image data of a treatment area (e.g., treatment area 350) (at 204), such as a treatment area of the spine. The method 200 may include, by the computing system 100, receiving a selection of at least one boney structure (at 206) in the treatment area, via a graphical user interface 500, as shown in FIG. 5, to which the model of the 3D bone-protecting drill guide device should be reverse-engineered surface.

The method 200 may include, by the computing system 100, receiving implant type data (at 208) and receiving implant location data associated the treatment area (at 210), such as using a graphical user interface (e.g., graphical user interfaces 600A and 600B as shown in FIGS. 6A-6B). The method 200 may include, by the computing system 100, generating a three-dimensional model of a 3D bone-protecting drill guide device for the planned surgery (at 212). The model of the 3D bone-protecting drill guide device may be displayed in a graphical user interface in a display device 115. During modeling (at 212, the computing system 100 may use ML algorithms to model a prosthetic of a boney structure that is missing or damaged, for example.

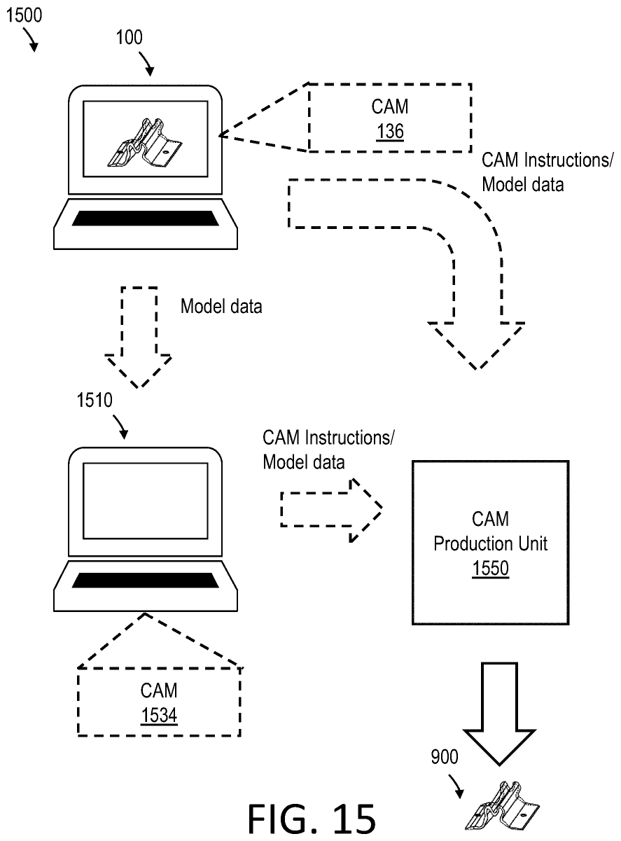

The method 200 may include, by the computing system 100, (optional) receiving input to add connectors to the 3D model of the 3D bone-protecting drill guide device (at 214), denoted in a dashed box. By way of non-limiting example, the ML algorithms or the user may select an apex of a protruding boney structure, such as a spinous process. The apex may be identified as the highest point of all boney structures in the image data. Before, adding the connectors, the divide the 3D model into halves or portions, such as along a sagittal plane, for example A pair of connectors may be added to the divided apex along the sagittal plane such that the connectors may be mated or connected together. The method 200 may include 3D printing the 3D bone-protecting drill guide device (at 216) using CAM software, such as by a 3D printer 1660 (FIG. 16) or CAM production unit 1550 (FIG. 15). The 3D printer may be connected to a different computing system or the computing system 100.

The method 200 may be implemented using hardware, firmware, software or a combination of any of these. For instance, method 200 may be implemented as part of a microcontroller, processor, and/or graphics processing units (GPUs) and an interface with a register, data store and/or memory device 1620 (FIG. 16) for storing data and programming instructions, which when executed, performs the steps of method 200 described herein.

The 3D geometry may be created by using extruded two-dimensional (2D) geometry. The 3D geometry may be represented as Nonuniform Rational B-Splines (NURBS).

FIGS. 8-12A-B illustrate example 3D bone-protecting drill guide devices 800, 900, 900B, 1000, 1100A, 1100B, 1200A, and 1200B. The 3D bone-protecting drill guide device 1000 is an example, device with a connector added. Based on the type of material used for the manufacture of the devices and the particular configuration of the honey structure, 3D bone-protecting drill guide device 800, for example, may be snapped onto the boney structure. In other embodiments, the 3D bone-protecting drill guide device may be split into halves or pieces that may be hinged together via a connector for ease of installation on the boney structures.

The 3D bone-protecting drill guide device may be configured to be secured to a boney structure without the need for bone-to-device fasteners and rely on the natural anatomical formation for locating a position to install the 3D bone-protecting drill guide device.

FIG. 8 is an exploded end view that illustrates an example 3D bone-protecting drill guide device 800 having at least one closed end and mounted on a boney structure of a cervical vertebra 80 of a cervical treatment area. The 3D bone-protecting drill guide device 800 has application as a cervical bone mount to protect cervical boney structures associate with cervical vertebrae. Cervical boney structures may be soft and small making it difficult to mount certain surgical instruments and operate to treat a spinal disease. In other scenarios, the cervical boney structure may be deformed such that the deformity makes it difficult to mount certain surgical instruments to perform a surgical implant. The 3D bone-protecting drill guide device 800 has application for the treatment for a spine deformity of an unknown spine curve that makes it difficult to mount a current “clamp” used in current surgical solutions.

The 3D bone-protecting drill guide device 800 may include a 3D body 802 of biocompatible material between the outer surface 804 and the interior surface 806 shown in dashed lines. The 3D bone-protecting drill guide device 800 may include implant guides 814 and 824, for example. Each implant guide 814 and 824 may be configured to extend from the outer surface 804 and through the body material and the interior surface 806. This may provide a window to a planned implant location relative to the at least one boney structure of the patient. Arrows D1 and D2 may represent the pre-planned direction for drilling into the vertebra 80, shown in dashed lines, to implant the bone constructs.

The interior surface 806 may be created based on a reverse-engineering surface from the image data of the patient to be treated, such as by generating a mesh of the anatomical surface in the image data. The interior surface 806 may be configured directly conform to the anatomical surface. The outer surface 804 or exterior surface may also conform to the interior surface 806 or the reverse-engineered surface from the image data with a solid volume 810 between the outer surface 804 and the interior surface 806 to form a wall or shell. The solid volume 810 is a thickness of the wall or shell. In some embodiments, the thickness may be varied depending on the amount of rigidity needed for mounted a surgical tool, if necessary. In some embodiments, the outer surface 804 and the interior surface 806 are separated by a hollow volume of space.

In various embodiments, the outer surface 804 may include end walls 820 such that the 3D printed bone-protecting drill guide device 800 encloses a selected boney structure, such as a spinous process.

FIG. 9A is a perspective view that illustrates an example 3D bone-protecting drill guide device 900 having at least one open end. The 3D bone-protecting drill guide device 900 may include a 3D body 902 of biocompatible material between the outer surface 904 and the interior surface 906 shown in dashed lines. The 3D bone-protecting drill guide device 900 may include implant guides 914 and 924, for example. Each implant guide 914 and 924 may be configured to extend from the outer surface 904 and through the body material and the interior surface 906. This may provide a window to a planned implant location relative to the at least one boney structure of the patient. The 3D bone-protecting drill guide device 900 may be installed on the vertebra 80 (FIG. 8). The device 900 include a guide body portion 915, which is part of the 3D body 902. The thickness 910 of the guide body portion 915 may be a function of the diameter of the drill implement. The implant guides 914 and 924 are holes formed in the guide body portion 915 to provide a window to the underlying boney structure. If thickness (t) is the thickness 910 and D is the diameter of the drill implement, then t=D×F, where F is in the range of 1-3. The window has a size and shape being pre-calculated as a function of a size of a pre-determined tool to be inserted in the window. The tool may be selected during the pre-planning phase to be inserted through the window down to a boney structure to form an implant hole in the bone under the implant guide.

In some embodiments, the 3D bone-protecting drill guide device 900 may be made of biocompatible material that may be resilient, may be slipped over the boney structure and snapped into place, in such an embodiments, the end 920 does not include end wall, such as shown in FIG. 8. By way of non-limiting example, the 3D printed bone-protecting drill guide devices 800 and 900 may be modeled to conform to the spinous process and lamina of a cervical vertebra, as shown in FIG. 8.

The 3D body 902 may include an implant guide protector drill guide pad 912 for forming the pre-planned implant guide 914 in the 3D bone-protecting drill guide device 900. The 3D body 902 may include an implant guide pad 922 for forming the pre-planned implant guide 924 in the 3D bone-protecting drill guide device 900.

FIG. 9B is a perspective view that illustrates an example 3D bone-protecting drill guide device 900B for protecting two adjacent vertebrae and having at least one open end. The 3D bone-protecting drill guide device 900B may include a 3D body 902B having a first-level 3D bone-protecting drill guide device 900¹ and a second-level 3D bone-protecting drill guide device 900². The 3D bone-protecting drill guide devices 900¹ and 900² may be similar to 3D bone-protecting drill guide device 900 previously described in relation to FIG. 9A. However, the 3D bone-protecting drill guide devices 900¹ and 900² may not be identical because each represents a 3D representation of the particular boney structures for the corresponding level of the vertebral column. For example, the 3D bone-protecting drill guide devices 900¹ may be smaller than the 3D bone-protecting drill guide devices 900² and may have a deformity.

It should be understood, the 3D bone-protecting drill guide device 900B may link together, via links 971 and 972, a 3D bone-protecting drill guide device for two or more levels of a vertebral column. In this example, there are two levels of 3D bone-protecting drill guide devices 900¹ and 900² linked together via parallel links 971 and 972. The length of the links 971 and 972 may vary based on the thickness of the intervertebral disc between any two levels of vertebrae. The length of the annulus fibrosus of the intervertebral disc may vary due to injury, disease or deformity, for example. The length of the links 971 and 972 may vary to conform to the length of the annulus fibrosus of the intervertebral disc so that the implant guides 914 and 924 may be aligned with the pre-planned implant locations for each vertebra.

In some embodiments, one of the 3D bone-protecting drill guide device 900¹ or the 3D bone-protecting drill guide devices 900² may be a prosthetic model of a missing boney structure.

FIG. 10 is a perspective end view that illustrates an example 3D bone-protecting drill guide device 1000 having a two-piece construction for a boney structure of a thoracic vertebra of a thoracic treatment area. FIG. 11A is a perspective view that illustrates an example 3D bone-protecting drill guide device 1100A having at least one open end for a boney structure of a thoracic vertebra of a thoracic treatment area. Since the 3D bone-protecting drill guide device 1100A is similar to the 3D bone-protecting drill guide device 900, only the differences will be described. In this example, the model of the 3D bone-protecting drill guide device 1100A is based on the thoracic vertebra of a patient and may installed with a slight press over the boney structure. The 3D bone-protecting drill guide device 1100A includes portions that would overlay on top of the vertebra lamina. FIG. 11B is a perspective view that illustrates an example 3D bone-protecting drill guide device 1100B of FIG. 11A with a robotic effector interface 1130. The robotic effector interface 1130 is configured to connect to a robotic end effector, for example, as will be described in relation to FIG. 14B. The robotic effector interface 1130 may include threads 1135 or other mechanisms for connection to the robotic end effector directly or indirectly. Instead of threads 1135, the interface 1130 may be configured to attach to a robotic grip or clamping mechanism.

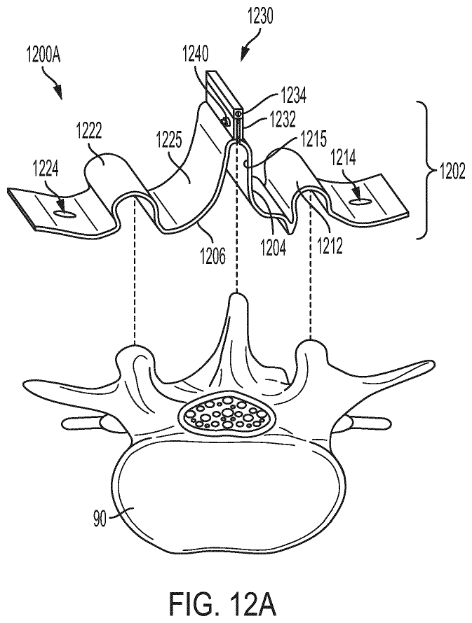

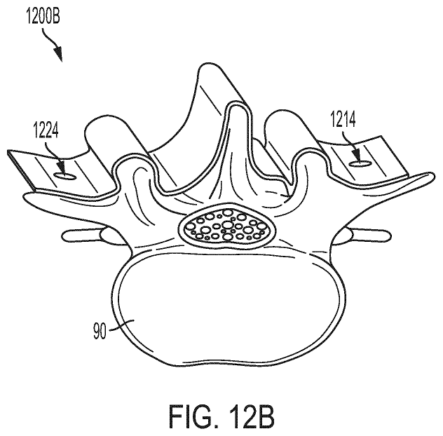

Referring again to FIG. 10, the 3D bone-protecting drill guide device 1000 may include a body 1002 having a two-piece construction. In this example, the body 1002 may be configured to model a boney structure of a thoracic vertebra of a thoracic treatment area. The 3D bone-protecting drill guide device 1000 may include a first portion 1015 and a second portion 1025 of a spinous process of the thoracic vertebra and lamina with the implant guides 1014 and 1024. The first portion 1015 and the second portion 1025 are separate body members. The computing system 100 may be configured to add connectors 1030 or interfaces to the model of the 3D bone-protecting drill guide device 1000 so that it may be installed. In this example, the connector 1030 may include a male interface 1034A and a female connector 1034B that will snap or fit together. In some embodiments, the connectors 1030 may include a fastener to lock or fix the portions together, as will be described in relation to FIG. 12A.

FIG. 12A is a perspective view that illustrates an example 3D bone-protecting drill guide device 1200A having a two-piece construction for a boney structure of a lumbar vertebra of a lumbar treatment area. The 3D bone-protecting drill guide device 1200A may extend to and slop along a portion of the transverse process of the thoracic vertebra. The 3D printed bone-protecting drill guide device may be modeled to conform to the spinous process, the lamina, the articular process and at least a portion of the transverse process of at least one lumbar vertebra, as shown in FIG. 12A. While, the 3D bone-protecting drill guide device 1200A is represented for a vertebra application, the two-piece construction of the 3D bone-protecting drill guide device may be configured to track any a boney structure, such as without limitation, boney structures of a knee or other orthopedic application to treat a joint. Any connectors for a two-piece construction may vary. Additionally, any interface for a robotic effector may change based on surgery procedure to treat a bone.

The 3D bone-protecting drill guide device 1200A may include a 3D body 1202 of biocompatible material between the outer surface 1204 and the interior surface 1206. The 3D bone-protecting drill guide device 1200A may include implant guides 1214 and 1224, for example. Each implant guide 1214 and 1224 may be configured to extend from the outer surface 1204 and through the body material and the interior surface 1206. The implants may want to be unobstructed entirely for easy interface, or to be well guided such as by using a robotic navigation system (FIG. 14A) to install the implants (i.e., bone fastener).

The 3D body 1202 may include an implant guide pad 1212 for forming the pre-planned implant guide 1214 in the 3D bone-protecting drill guide device 1200A. The 3D body 1202 may include an implant guide pad 1222 for forming the pre-planned implant guide 1224 in the 3D bone-protecting drill guide device 1200A.

The body 1202 may have a two-piece construction. In this example, the body 1202 may be configured to model a boney structure of at least one lumbar vertebra of a lumbar treatment area. The 3D bone-protecting drill guide device 1200A may include a first portion 1215 and a second portion 1225 of a spinous process of the at least one lumbar vertebra and lamina with the implant guides 1214 and 1224. The computing system 100 may be configured to add connectors 1230 or interfaces to the model of the 3D bone-protecting drill guide device 1200A so that it may be installed on at least one vertebra. In this example, the connector 1230 may include supports 1232 coupled to a hinge member 1234. The hinge member 1234 may allow one of the first portion 1215 and the second portion 1225 to pivot or rotate relative to the other. In some embodiments, the fastener 1240 may be used to lock or fix the position of the first portion 1215 and the second portion 1225 once installed. The connector 1230 may have formed therein a hole for receipt of the fastener 1240.

FIG. 12B is a perspective view that illustrates an example 3D bone-protecting drill guide device 1200B having a one-piece construction installed on a boney structure of a lumbar vertebra 90. The 3D bone-protecting drill guide device 1200A may include implant guides 1214 and 1224. In this example, the one-piece construction may be installed by a surgeon or assistant. In other embodiments, the one-piece construction may include connectors or a robotic effector interface, such as interface 1130, or other interface for attachment of robotic grips, for example.

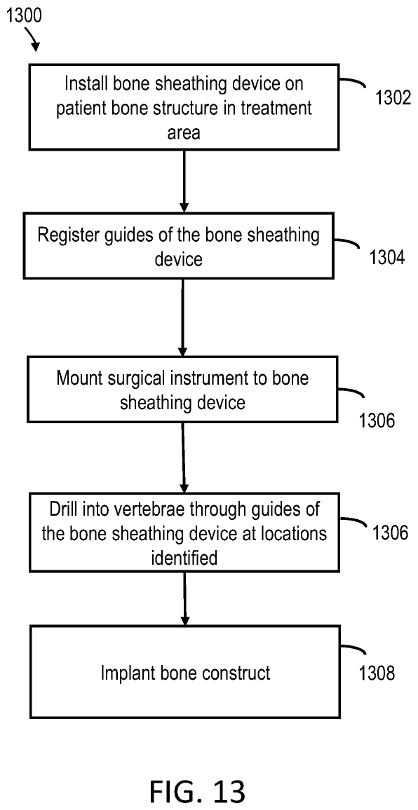

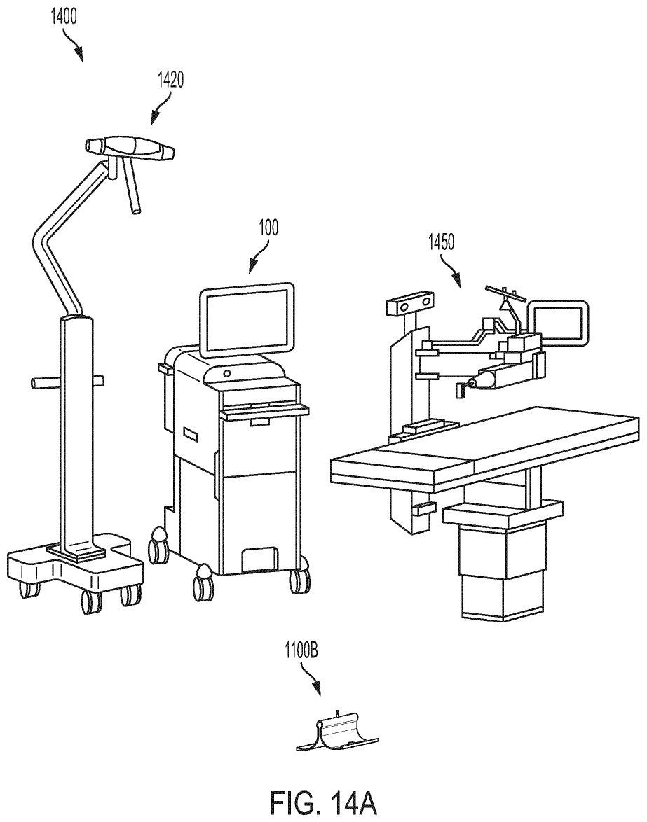

FIG. 13 is a flowchart that illustrates a method 1300 of performing surgery using the 3D bone-protecting drill guide device. The surgery may be performed using the surgery implant system 1400 (FIG. 14A) or other robotic surgery system. The surgery may be performed by a surgeon without the aid of a robotic surgery system, in some embodiments.

The method 1300 may include installing at least one 3D bone-protecting drill guide device on at least one boney structure in a treatment area (at 1302). The method 1300 may include registering at least one pre-planned implant guide of the 3D bone-protecting drill guide device (at 1304) to the location selected during the pre-planning phase of the surgery. The method 1300 may include mounting a surgical instrument to the bone-protecting drill guide device (at 1306) and drilling into a bone or vertebra though the implant guide (at 1308). The method 1300 may include implanting at least one bone construct using at least one drilled hole (at 1310). After drilling the holes, the 3D bone-protecting drill guide device may be removed from the patient before closing the incisions of the patient. In other embodiments, the 3D bone-protecting drill guide device may remain implanted in human tissue, after the surgery is complete.

FIG. 14A is a diagram that illustrates a surgical implant system 1400. The surgical implant system 1400 may include a computing system 100 as described above in FIG. 1 that may be configured to generate a 3D model of a 3D bone-protecting drill guide device, based on pre-operative imaging data of a patient. The surgical implant system 1400 may include robotic guidance system 1450 and a tracking system 1420, such as a Mazor X Stealth™ Edition robotic guidance system, by Medtronic Sofamor Danek USA, Inc. The surgical implant system 1400 may include at least one 3D bone-protecting drill guide device 900, for example.

It should be understood, that a 3D bone-protecting drill guide device (e.g., 3D bone-protecting drill guide device 900) may be used with other surgery implant systems including those that do not employ robotics. The 3D bone-protecting drill guide device has application in any surgery in which one or more of: 1) a boney structure may be used to mount or clamp a surgical instrument; 2) a boney structure may be subject to impact forces due to use of a surgical instrument during surgery; 3) a boney structure may be soft or brittle; 4) a boney structure is missing for mounting or clamping an instrument needed to perform the surgery; 5) registration of multiple implant sites is required; and 6) there is an insufficient area available to mount surgical instruments to perform a revision surgery.

The 3D bone-protecting drill guide device has application for pediatric surgery. Pediatric bones can be soft and small that makes it difficult to mount a clamp, could be break, unknown segment shape. The 3D bone-protecting drill guide device may be used instead of the robotic arm by making a drill guide inside the 3D bone-protecting drill guide device.

FIG. 14B is a diagram that illustrates a robotic arm 1455 and end effector 1460 of the robotic guidance system 1450 for connection to the 3D bone-protecting drill guide device 1100B of FIG. 11B. The robotic arm 1455 may be supported by upright-support member 1453. The end effector 1460 may connect to an effector extender 1465 configured to be coupled to end effector 1460. The effector extender 1465 may be configured to be coupled to the robotic effector interface 1130. The effector extender 1465 may be guided to place the 3D bone-protecting drill guide device 1100B in place over the appropriate bone, such as shown in FIG. 12B.

FIG. 15 is a flow diagram that illustrates a method 1500 for the production of a 3D bone-protecting drill guide device by a CAM production unit 1550. The CAM production unit 1550 may be a 3D printer or other computer-aided production unit. The method 1500 may include, by the computing system 100 (FIG. 1), communicating model data, such as CAD model data, representative of the 3D bone-protecting drill guide device to a computing device 1510 of a vendor. The computing device 1510 may include an operation system and CAM applications 1534 configured to interface or drive the CAM production unit 1550. After receiving the CAD model data, the computing device 1510 may communicate CAM instructions and the model data to the CAM production unit 1550. The method 1500 may include, by the production unit 1550, manufacturing at least one the 3D bone-protecting drill guide device 900, for example, such as without limitation, by 3D printing techniques, based on the received model data and instructions.

Alternately, the method 1500 may include, by the computing system 100, communicating the CAM instructions and model data representative of the 3D bone-protecting drill guide device to the CAM production unit 1550. The computing system 100 may include a CAM application 136 or may access a CAM application from a remote server or cloud computing device. In various embodiments, the method 1500 may include, by the CAM production unit 1550, manufacturing the 3D bone-protecting drill guide device, based on received the model data and instructions.

FIG. 16 depicts an example of internal hardware that may be included in any of the electronic components of an electronic device 1600 as described in this disclosure such as, for example, a computing device, a remote server, cloud computing system and/or any other integrated system and/or hardware that may be used to contain or implement program instructions.

A bus 1610 serves as the main information highway interconnecting the other illustrated components of the hardware. Processor(s) 1605 may be the central processing unit (CPU) of the computing system, performing calculations and logic operations as may be required to execute a program. CPU 1605, alone or in conjunction with one or more of the other elements disclosed in FIG. 16, is an example of a processor as such term is used within this disclosure. Read only memory (ROM) and random access memory (RAM) constitute examples of tangible and non-transitory computer-readable storage media, memory devices 1620 or data stores as such terms are used within this disclosure. The memory device 1620 may store an operating system (OS) of the computing device, a server or for the platform of the electronic device.

Program instructions, software or interactive modules for providing the interface and performing any querying or analysis associated with one or more data sets may be stored in the computer-readable storage media (e.g., memory device 1620). Optionally, the program instructions may be stored on a tangible, non-transitory computer-readable medium such as a compact disk, a digital disk, flash memory, a memory card, a universal serial bus (USB) drive, an optical disc storage medium and/or other recording medium.

An optional display interface 1630 may permit information from the bus 1610 to be displayed on the display device 1635 in audio, visual, graphic or alphanumeric format. Communication with external devices may occur using various communication ports 1640. A communication port 1640 may be attached to a communications network, such as the Internet or an intranet. In various embodiments, communication with external devices may occur via one or more short range communication protocols. The communication port 1640 may include communication devices for wired or wireless communications and may communicate with a 3D printer 1660 or other CAM production unit 1550 (FIG. 15).

The hardware may also include a user interface 1645, such as a graphical user interface (GUI), that allows for receipt of data from input devices, such as a keyboard 112 (FIG. 1) or other input device 1650 such as a mouse, a joystick, a touch screen, a remote control, a pointing device, a video input device and/or an audio input device. The GUIs, described herein, may be displayed using a browser application being executed by an electronic device and/or served by a server (not shown). For example, hypertext markup language (HTML) may be used for designing the GUI with HTML tags to the images of the patient and other information stored in or served from memory of the server (not shown).

In this document, “electronic communication” refers to the transmission of data via one or more signals between two or more electronic devices, whether through a wired or wireless network, and whether directly or indirectly via one or more intermediary devices. Devices are “communicatively connected” if the devices are able to send and/or receive data via electronic communication.

In one or more examples, the described techniques and methods may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

What is claimed is:

1. A surgical bone-protecting drill guide device, comprising:
a body formed of biocompatible material forming a shell, the body including:
an outer surface,
an interior surface being a reverse-engineering surface approximation of a protruding boney structure of one or more bones in an image of a patient,
body material between the outer surface and the interior surface, and implant guides,
wherein each implant guide is configured to extend from the outer surface and through the body material and the interior surface and provide a window to a pre-planned implant location for implanting a respective one implant relative to the protruding boney structure of the patient, and
wherein the window has a size and shape pre-calculated as a function of a size of a pre-determined tool to be inserted through the window.

2. The device of claim 1, wherein:
the outer surface is the reverse-engineering surface approximation of the protruding boney structure; and
the body material fills a space between the outer surface and the interior surface.

3. The device of claim 1, wherein the body comprises a first portion and a second portion, the first portion and the second portion are separate body members; and
further comprising:
a first connector coupled a top side of the first portion, and
a second connector coupled to a top side of the second portion,
wherein the first connector and the second connector are configured to connect together.

4. The device of claim 3, wherein the body further comprises holes in the first portion and the second portion; and
further comprising:
a fastener coupled to the first connector and the second connector via the holes, the fastener configured to lock the first portion and the second portion about the boney structure.

5. The device of claim 1, wherein:
a boney structure to which the implant is to be implanted is adjacent to the protruding boney structure; and
the body further comprises:
at least one adjacent boney structure protector, the at least one adjacent boney structure protector includes:
an outer surface portion of the outer surface,
an interior surface portion of the interior surface, the interior surface portion is a reverse-engineering surface approximation of the at least one adjacent boney structure of the patient, and
body material between the outer surface portion and the interior surface portion; and
each implant guide extends through the outer surface portion and the interior surface portion.

6. The device of claim 5, wherein the protruding boney structure comprises boney structures of a plurality of vertebrae.

7. The device of claim 1, wherein the body is configured to be slipped over the protruding boney structure.

8. The device of claim 1, wherein:
the protruding boney structure comprises at least one of:
a spinous process,
a transverse process,
an articular process,
an inferior articular process, and
a superior articular process; and
the protruding boney structure is adjacent to at least one boney structure including a vertebra lamina.

9. A method, comprising:
receiving, by a computing system, pre-operative image data of at least one bone with a protruding boney structure of a patient;
receiving, by the computing system, pre-planned implant location data of pre-planned implant locations at which implants are to be implanted relative to the protruding boney structure of the at least one bone; and
modeling, by the computer system, a body of a three-dimensional bone-protecting drill guide device by:
forming an interior surface as a reverse-engineering surface approximation of the protruding boney structure of the patient,
forming an outer surface having a predetermined thickness from the interior surface, and
forming implant guides,
wherein each implant guide is configured to extend from the outer surface and through the body and the interior surface and the implant guides provide a window to the pre-planned implant location for implanting a respective one implant relative to the protruding boney structure of the patient, and
wherein the window has a size and shape pre-calculated a function of a size of a pre-determined tool to be inserted through the window.

10. The method of claim 9, wherein:
the outer surface is the reverse-engineering surface approximation of the protruding boney structure.

11. The method of claim 9, wherein the modeling, by the computer system, of the body of the three-dimensional bone-protecting drill guide device, further comprises modeling by:
determining an apex of the reverse-engineering surface approximation of the protruding boney structure of the patient;
forming a first portion and a second portion of the body along a plane of the protruding boney structure, the first portion and the second portion are separate body members;
forming a first connector coupled a top side of the first portion; and
forming a second connector coupled to a top side of the second portion,
wherein the first connector and the second connector are configured to connect together.

12. The method of claim 11, wherein the modeling, by the computer system, of the body of the three-dimensional bone-protecting drill guide device, further comprises modeling by forming a hole in each of the first connector and the second connector, the hole adapted to receive a fastener to lock the first portion and the second portion about the protruding boney structure.

13. The method of claim 9, wherein:
the pre-operative image data of the at least one bone further comprises at least one boney structure adjacent to the protruding boney structure;
the modeling, by the computer system, of the body of the three-dimensional bone-protecting drill guide device, further comprises modeling by:
forming at least one adjacent boney structure protector, the at least one adjacent boney structure protector being formed by:
forming an interior surface portion being a reverse-engineering surface approximation of the at least one adjacent boney structure of the patient, and
forming an outer surface portion with a thickness from the interior surface portion; and
each implant guide extends through the outer surface portion and the interior surface portion.

14. The method of claim 13, wherein the protruding boney structure comprises a boney structure of a plurality of vertebrae.

15. The method of claim 9, wherein the body is configured to be slipped over the protruding boney structure of the patient.

16. The method of claim 9, wherein:
the protruding boney structure comprises at least one of:
a spinous process,
a transverse process,
an articular process,
an inferior articular process, and
a superior articular process.

17. A method, comprising:
providing a bone-protecting drill guide device of claim 1 for a protruding boney structure of a patient;
installing the bone-protecting drill guide device on the protruding boney structure of the patient;
registering a location of an implant guide;
mounting a surgical instrument to the installed bone-protecting drill guide device; and
drilling a hole for a bone construct using the implant guides using the mounted surgical instrument.

18. The method of claim 17, further comprising:
using a robotic surgical system to install the bone construct in each drilled hole.

19. The method of claim 17, wherein the bone-protecting drill guide device comprises:
a first portion,
a second portion, and
a connector; and
the method further comprising:
installing the first portion on the protruding boney structure of the patient, and
connecting the second portion to the first portion, via the connector.

20. The method of claim 17, wherein:
the protruding boney structure comprises at least one of:
a spinous process;
a transverse process;
an articular process;
an inferior articular process; and
a superior articular process.
